(19) United States
(12) Patent Application Publication
(10) Pub. No.: US 2005/0043220 A1
(43) Pub. Date: Feb. 24, 2005

(54) Methods and compositions for treating macular degeneration

(76) Inventors: David R. Guyer, New York, NY (US); Denis O'Shaughnessy, Montville, NJ (US)

Correspondence Address: EYETECH PHARMACEUTICALS, INC., 3 TIMES SQUARE 12TH FLOOR, NEW YORK, NY, 10036, US

(21) Appl. No.: 10/928,533

(22) Filed: Aug. 27, 2004

Related U.S. Application Data
(63) Continuation-in-part of application No. 10/291,091, filed on Nov. 8, 2002.
(60) Provisional application No. 60/498,746, filed on Aug. 28, 2003.

(51) Int. Cl. A61K 38/16; C08G 63/48; A61K 38/18

(52) U.S. Cl. 514/7; 530/352; 525/54.1

(57) ABSTRACT

This invention relates to methods of treating age-related macular degeneration (AMD). In particular, this invention provides methods of treating all forms of wet, age-related macular degeneration. The method of the invention is directed to the administration of an anti-vascular endothelial growth factor (anti-VEGF) compound to treat wet AMD.

### RELATED APPLICATIONS

This application is a continuation-in-part of U.S. Ser. No. 10/291,091, filed Nov. 8, 2002 and claims benefit of U.S. provisional application 60/498,746, filed Aug. 28, 2003, the disclosures of which are hereby incorporated in their entirety by reference.

### FIELD OF THE INVENTION

This invention relates to methods of treating age-related macular degeneration (AMD). In particular, this invention provides methods of treating all forms of wet, age-related macular degeneration. The method of the invention is directed to the administration of an anti-vascular endothelial growth factor (anti-VEGF) compound to treat wet AMD

### BACKGROUND OF THE INVENTION

The National Eye Institute and Prevent Blindness America estimated that in 2002, approximately 3.4 million Americans age 40 and older were visually impaired, with over one million being legally blind. See Prevent Blindness America and National Eye Institute, Vision Problems in the U.S. (2002). The prevalence of blindness and vision impairment increases rapidly as people age, particularly in the over-75 age group. According to the National Center for Health Statistics, in 1997, 26% of all nursing home residents in the United States, totaling over 420,000 individuals, had some level of visual impairment. See National Center for Health Statistics, National Nursing Home Survey (1997), available at http://www.cdc.gov/nchs. As a result of demographic changes in the United States, the number of individuals with vision impairment is expected to double in the next three decades. See Prevent Blindness America and National Eye Institute, Vision Problems in the U.S.

Vision impairment causes personal trauma and incapacity, thereby imposing large costs upon society. A study performed by J. M. McNeil in 2001 found that among persons in the United States between the ages of 21 and 64, only 41.5% of persons with visual impairment were employed, as compared to 84% of persons without any disabilities. See U.S. Bureau of the Census, Current Population Reports, P70-61 & P70-73 (2001). The same study found that the average annual earnings of individuals with visual impairment were approximately 31% less than those of persons without any disabilities. See id. In 1998, the National Advisory Eye Council estimated that the economic impact of visual disorders and disabilities in the United States was more than $38.4 billion per year, with $22.3 billion of that amount attributed to direct costs and another $16.1 billion attributed to indirect costs.

Eye disease can be caused by many factors and can affect both the front and back of the eye. In its most extreme cases, eye disease can result either in partial blindness, in which some vision is preserved, or in total blindness. AMD and diabetic retinopathy, including DME, are among the leading causes of significant vision loss. See Prevent Blindness America and National Eye Institute, Vision Problems in the U.S. These diseases deny patients their sight, and, as a result, their ability to live independently and perform daily activities.

AMD is the leading cause of irreversible, severe blindness in patients over the age of 55 in the western world, and affects almost 15 million people in the United States alone. See American Macular Degeneration Foundation, available at http://www.macular.org; Klein et al., Prevalence of Age-related Maculopathy, The Beaver Dam Eye Study, 99 Ophthalmol. 933-43 (1992); Schepens Eye Research Institute, Macular Degeneration Fact Sheet; U.S. Bureau of the Census, 1998 Population Estimates (1998). AMD is caused by the deterioration of the central portion of the retina, known as the macula. There are two types of AMD: dry AMD and wet AMD. While many more people suffer from dry AMD, it accounts for only 10% of the severe vision loss associated with AMD and has no generally accepted treatment. See National Eye Institute, available at http://www.nei.nih.gov. On the other hand, wet AMD is responsible for 90% of the severe vision loss associated with this disease. See id.

There are three subtypes of the wet form of AMD: predominantly classic (affecting approximately 25% of patients suffering from wet AMD), minimally classic (affecting approximately 35% of wet AMD sufferers) and occult (affecting approximately 40% of wet AMD sufferers). See QLT, Inc., available at http://www.qltinc.com/Qltinc/main/mainhome.cfm. Although the specific factors that cause wet AMD are not conclusively known, aging appears to be the most important risk factor. The number of cases of wet AMD will increase significantly as baby boomers age and overall life expectancy increases.

Research of wet AMD shows that vascular endothelial growth factor (“VEGF”) is one of the major factors causing both abnormal blood vessel growth (angiogenesis) and blood vessel leakage in the eye. Specifically, preclinical studies have shown that a) in multiple animal species, including humans, and models, VEGF levels are elevated around growing and leaky blood vessels, b) blocking VEGF results in the prevention and regression of these abnormal vessels in primates and other species and c) VEGF alone is sufficient to trigger the abnormal blood vessel growth that characterizes wet AMD and the blood vessel leakage that characterizes DME. See A. P. Adamis et al., Inhibition of vascular endothelial growth factor prevents retinal ischemia-associated iris neovascularization in a nonhuman primate, 114(1) Arch. Ophthalmol. 66-71 (1996); A. Kvanta et al., Subfoveal fibrovascular membranes in age-related macular degeneration express vascular endothelial growth factor, 37 Invest. Ophthalmol. Vis. Sci. 1929-34 (1996); G. Lutty et al., Localization of vascular endothelial growth factor in human retina and choroids, 114 Arch. Ophthalmol. 971-77 (1996); M. J. Tolentino et al., Intravitreous injections of vascular endothelial growth factor produce retinal ischemia and microangiopathy in an adult primate, 103(11) Ophthalmology 1820-28 (1996); M. J. Tolentino, Vascular endothelial growth factor is sufficient to produce iris neovascularization and neovascular glaucoma in a nonhuman primate, 114(8) Arch. Ophthalmol. 964-70 (1996).

Substantial peer-reviewed research has found high concentrations of VEGF in the eyes of humans afflicted with wet AMD. For example, in a study published by the New England Journal of Medicine, vitreous levels of VEGF were shown to be very high in patients with angiogenic diseases, but were negligible in patients undergoing the same type of surgery for nonangiogenic diseases. See Aiello et al., 331 New. Eng. J. Med. 1480-87 (1994). In a separate study, it was shown that ocular VEGF levels are elevated in patients with active DME. See S. A. Vinores et al., Upregulation of vascular endothelial growth factor in ischemic and non-ischemic human and experimental retinal disease, 12(1) Histol. Histopathol. 99-109 (1997).

Macugen™, (pegaptanib sodium), a pegylated anti-VEGF aptamer, is described in greater detail in U.S. Pat. Nos. 6,426,335 and 6,051,698, hereby incorporated in their entirety by reference. It blocks blood vessel growth and inhibits neovascularization in preclinical models. Macugen™ has been shown in preclinical studies to have antipermeability properties, which prevent blood vessels from leaking. Such leakage causes the macula to become edematous and impairs vision.

One Phase 1 and two Phase 2 clinical trials of Macugen™ as a treatment for wet AMD were completed in June 2001. In the Phase 1 trial, 15 patients received varying doses of Macugen™. The trial showed that the therapy was well tolerated. Approximately 80% of the patients showed stabilized or improved vision, and approximately 26.7% of the patients demonstrated the ability to read three lines or more on a standard vision chart as compared to baseline vision.

In the first Phase 2 trial of ten patients, the therapy was also well tolerated. Approximately 87.5% of the patients in this trial showed stabilized or improved vision, with approximately 25.0% of the patients demonstrating the ability to read three lines or more on a standard vision chart compared to baseline vision.

In the second Phase 2 trial, an additional 11 patients received Macugen™. This trial consisted solely of patients with the predominantly classic form of wet AMD who were also receiving photodynamic therapy. As in the other trials, the therapy was well tolerated. Approximately 90% of the patients in this study showed stabilized or improved vision, with approximately 60% of the patients demonstrating the ability to read three lines or more on a standard vision chart compared to baseline vision. Furthermore, the need for retreatment with photodynamic therapy at the end of three months was reduced from 93% to 20% when photodynamic therapy was administered in combination with Macugen™.

These Phase 1 and Phase 2 clinical trials were conducted on a relatively small number of patients, and there were no randomized controls. While there was no long term follow up for the Phase 1 trial patients, follow up was performed for the Phase 2 patients for 12 months.

Currently, Novartis AG's photodynamic therapy Visudyne® (verteporfin) is the only pharmaceutical based treatment for wet AMD approved by the FDA. However, it is only approved for patients with the predominantly classic form of wet AMD, and therefore may only be used to treat approximately 25% of all persons suffering from wet AMD. Historically, over 90% of cases treated with Visudyne® recurred within three months of the initial therapy and, therefore, required retreatment. See Photodynamic Therapy of Subfoveal Choroidal Neovascularization in Age-Related Macular Degeneration with Verteporfin: One-Year Results of 2 Randomized Clinical Trials—Tap Report 1, 117 Arch. Ophthalmol. 1329-45 (1999); QLT, Inc., available at http://www.qltinc.com/Qltinc/main/mainhome.cfm. In addition, 1% to 5% of photodynamic therapy patients experience severe vision loss following therapy, although some of these patients recover the lost vision over time.

In view of the deficiencies of existing therapies, a strong need remains for an effective treatment which can be used for all forms (occult, minimally classic, and predominantly classic) of wet-AMD.

### SUMMARY OF THE INVENTION

The present invention provides a method of treating all types of exudative age related macular degeneration comprising administering an anti-VEGF agent locally into the eye. In some embodiments, the anti-VEGF agent is an anti-VEGF aptamer and is administered at a dosage of about 0.1 mg-about 1.0 mg locally into the eye, wherein the treatment is effective to treat occult, minimally classic, and predominantly classic forms of wet macular degeneration. In some embodiments, the anti-VEGF aptamer is administered by intravitreal injection. In some embodiments, the anti-VEGF aptamer is administered every 4-6 weeks, and in other embodiments, the treatment is continued for a period of at least one year. In a particular embodiment, the anti-VEGF aptamer is PEGylated.

According to one embodiment, the present invention provides a method for treating macular degeneration comprising administering a therapeutically effective amount of an anti-VEGF agent locally into the eye wherein the treatment is effective to treat occult, minimally classic, and predominantly classic forms of wet macular degeneration, wherein the agent is an aptamer, antibody or antibody fragment.

According to a further embodiment, a method of treating macular degeneration is provided comprising administering an anti-VEGF agent locally into the eye, the agent being an aptamer, antibody, or antibody fragment, in an amount effective to achieve a maximum concentration of the agent in plasma of less than about 8 ng/ml wherein the treatment is effective to treat occult, minimally classic, and predominantly classic forms of wet macular degeneration.

According to another aspect, the invention provides a pharmaceutical formulation comprising an anti-VEGF aptamer conjugated to a polyethylene glycol in a pharmaceutically acceptable carrier formulation for local administration into the eye, wherein the aptamer is present in the formulation at a concentration of 0.1 to 3.0 mg/ml. According to one embodiment, the carrier comprises sodium phosphate and sodium chloride. According to one specific embodiment the carrier comprises 10 mM sodium phosphate and 0.9% sodium chloride.

According to another embodiment, the anti-VEGF agent is administered by intravitreous injection every 4-6 weeks for a period of at least one year and the anti-VEGF agent is an aptamer. The aptamer is conjugated to polyethylene glycol having a molecular weight of about 10-80 Kd or 20-45 Kd.

According to a further embodiment, the anti-VEGF agent is

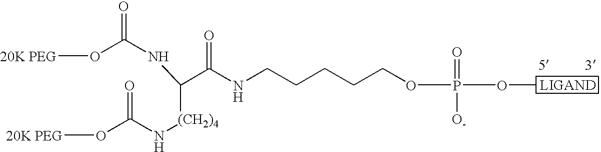

Ligand Component=fCmGmGrArAfUfCmAmGfUmGmAmAfUmGfCfUfUmAfUmAfCmAfUfCfCmG-3′3′−(VEGF ligand)

and the therapeutically effective amount is about 0.1-3.0 mg, 0.1-1.0 mg, or about 0.3 mg.

### Definitions

By “phototherapy” is meant any process or procedure in which a patient is exposed to a specific dose of light of a particular wavelength, including laser light, in order to treat a disease or other medical condition.

By “photodynamic therapy” or “PDT” is meant any form of phototherapy that uses a light-activated drug or compound, referred to herein as a photosensitizer, to treat a disease or other medical condition characterized by rapidly growing tissue, including the formation of abnormal blood vessels (i.e., angiogenesis). Typically, PDT is a two-step process that involves local or systemic administration of the photosensitizer to a patient followed by activation of the photosensitizer by irradiation with a specific dose of light of a particular wavelength. Photodynmaic therapies and photosensitizers are known in the art, as disclosed, for example, in U.S. Pat. Nos. 5,756,541, 5,798,349, 6,599,891, and 6,610,670 and PCT Publications WO 00/00204, WO 00/73308, WO 01/74818, WO 02/096366, WO 02/096417, WO 03/028629, WO 03/028628, WO 02/062386, WO 03/045432, and WO 01/58240, which are hereby incorporated in their entirety by reference.

By “anti-VEGF agent” is meant a compound that inhibits the activity or production of vascular endothelial growth factor (“VEGF”).

By “photosensitizes” or “photoactive agent” is meant a light-absorbing drug or other compound that upon exposure to light of a particular wavelength becomes activated thereby promoting a desired physiological event, e.g., the impairment or destruction of unwanted cells or tissue.

By “thermal laser photocoagulation” is meant a form of photo-therapy in which laser light rays are directed into the eye of a patient in order to cauterize abnormal blood vessels in the eye to seal them from further leakage.

By “effective amount” is meant an amount sufficient to treat a symptom of an ocular neovascular disease.

The term “light” as used herein includes all wavelengths of electromagnetic radiation, including visible light. Preferably, the radiation wavelength is selected to match the wavelength(s) that excite(s) the photosensitizer. Even more preferably, the radiation wavelength matches the excitation wavelength of the photosensitizer and had low absorption by non-target tissues.

### DETAILED DESCRIPTION OF THE INVENTION

According to the present invention, it has surprisingly been discovered that anti-VEGF treatment is effective in all types of wet-AMD. Further, treatment of wet-AMD in accordance with the teachings of the present invention provides superior results for the currently untreated population suffering from minimally classic and occult forms of this disease. Additionally, a dose regimen for providing effective treatment of all forms of wet-AMD is provided.

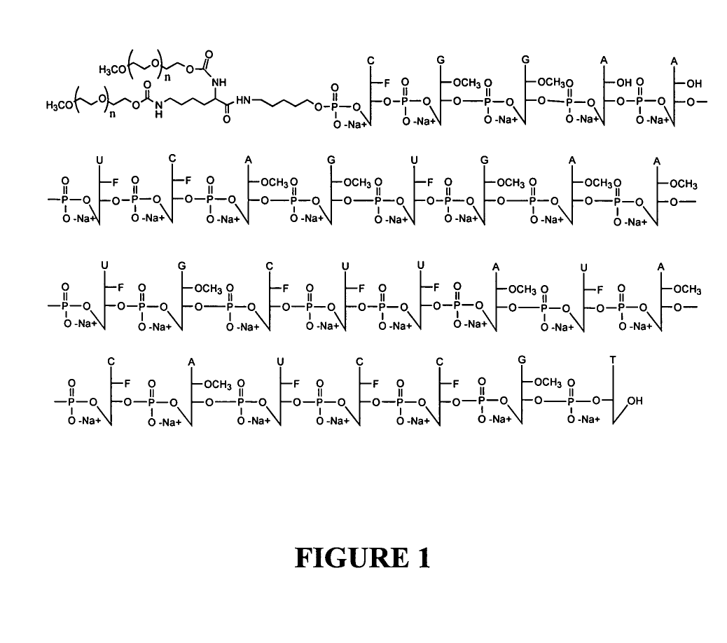

A variety of anti-VEGF therapies that inhibit the activity or production of VEGF, including aptamers and VEGF antibodies, are available and can be used in the methods of the present invention. The preferred anti-VEGF agents are nucleic acid ligands of VEGF, such as those described in U.S. Pat. Nos. 6,168,778 B1; 6,147,204; 6,051,698; 6,011,020; 5,958,691; 5,817,785; 5,811,533; 5,696,249; 5,683,867; 5,670,637; and 5,475,096, hereby incorporated in their entirety by reference. A particularly preferred anti-VEGF agent is pegaptanib sodium (EYE001, previously referred to as NX1838), which is a modified, pegylated aptamer that binds with high affinity to the major soluble human VEGF isoform and has the general structure shown in FIG. 1 (described in U.S. Pat. No. 6,168,788; Journal of Biological Chemistry, Vol. 273(32): 20556-20567 (1998); and In Vitro Cell Dev. Biol. Animal Vol. 35:533-542 (1999)).

Alternatively, the anti-VEGF agents may be, for example, VEGF antibodies or antibody fragments, such as those described in U.S. Pat. Nos. 6,100,071; 5,730,977; and WO 98/45331. Other suitable anti-VEGF agents or compounds that may be used in combination with anti-VEGF agents according to the present invention include, but are not limited to, antibodies specific to VEGF receptors (e.g., U.S. Pat. Nos. 5,955,311; 5,874,542; and 5,840,301); compounds that inhibit, regulate, and/or modulate tyrosine kinase signal transduction (e.g., U.S. Pat. No. 6,313,138 B1); VEGF polypeptides (e.g., U.S. Pat. No. 6,270,933 B1 and WO 99/47677); oligonucleotides that inhibit VEGF so expression at the nucleic acid level, for example antisense RNAs (e.g., U.S. Pat. Nos. 5,710,136; 5,661,135; 5,641,756; 5,639,872; and 5,639,736); retinoids (e.g., U.S. Pat. No. 6,001,885); growth factor-containing compositions (e.g., U.S. Pat. No. 5,919,459); antibodies that bind to collagens (e.g., WO 00/40597); and various organic compounds and other agents with angiogenesis inhibiting activity (U.S. Pat. Nos. 6,297,238 B1; 6,258,812 B1; and 6,114,320).

The anti-VEGF agents can also be administered topically, for example, by patch or by direct application to the eye, or by iontophoresis. The anti-VEGF agents may be provided in sustained release compositions, such as those described in, for example, U.S. Pat. Nos. 5,672,659 and 5,595,760. The use of immediate or sustained release compositions depends on the nature of the condition being treated. If the condition consists of an acute or over-acute disorder, treatment with an immediate release form will be preferred over a prolonged release composition. Alternatively, for certain preventative or long-term treatments, a sustained released composition may be appropriate.

The anti-VEGF agent may also be delivered using an intraocular implant. Such implants may be biodegradable and/or biocompatible implants, or may be non biodegradable implants. The implants may be permeable or impermeable to the active agent, and may be inserted into a chamber of the eye, such as the anterior or posterior chambers or may be implanted in the sclera, transchoroidal space, or an avascularized region exterior to the vitreous. In a preferred embodiment, the implant may be positioned over an avascular region, such as on the sclera, so as to allow for transcleral diffusion of the drug to the desired site of treatment, e.g. the intraocular space and macula of the eye. Furthermore, the site of transcleral diffusion is preferably in proximity to the macula.

Examples of implants for delivery of an anti-VEGF agent include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079, 5,902,598; 5,904,144; 5,916,584; 6,001,386; 6,074,661; 6,110,485; 6,126,687; 6,146,366; 6,251,090; and 6,299,895, and in WO 01/30323 and WO 01/28474, all of which are incorporated herein by reference.

Dosage levels on the order of about 1 ug/kg to 100 mg/kg of body weight per administration are useful in the treatment of neovascular disorders. When administered directly to the eye, the dosage range is about 0.3 mg to about 3 mg per eye, in some embodiments the dosage range is about 0.1 mg to about 1.0 mg per eye. The dosage may be administered as a single dose or divided into multiple doses. In general, the desired dosage should be administered at set intervals for a prolonged period, usually at least over several weeks, although longer periods of administration of several months or more may be needed.

According to another embodiment, the present invention features a method for treating a patient suffering from an ocular neovascular disease, which method includes the following steps: (a) administering to the patient an effective amount of an anti-VEGF aptamer; and (b) providing the patient with phototherapy, such as photodynamic therapy or thermal laser photocoagulation.

In one embodiment of the invention, the photodynamic therapy (PDT) includes the steps of: (i) delivering a photosensitizer to the eye tissue of a patient; and (ii) exposing the photosensitizer to light having a wavelength absorbed by the photosensitizer for a time and at an intensity sufficient to inhibit neovascularization in the patient's eye tissue. A variety of photosensitizers may be used, including but not limited to, benzoporphyrin derivatives (BPD), monoaspartyl chlorine, zinc phthalocyanine, tin etiopurpurin, tetrahydroxy tetraphenylporphyrin, and porfimer sodium (PHOTOFRIN), and green porphyrins.

In a related aspect, the present invention provides a method for treating an ocular neovascular disease in a patient, which method involves administering to the patient: (a) an effective amount of an anti-VEGF aptamer; and (b) a second compound capable of diminishing or preventing the development of unwanted neovasculature. The anti-VEGF agents or other compounds that may be combined with anti-VEGF aptamers include, but are not limited to: antibodies or antibody fragments specific to VEGF; antibodies specific to VEGF receptors; compounds that inhibit, regulate, and/or modulate tyrosine kinase signal transduction; VEGF polypepides; oligonucleotides that inhibit VEGF expression at the nucleic acid level, for example antisense RNAs; retinoids; growth factor-containing compositions; antibodies that bind to collagens; and various organic compounds and other agents with angiogenesis inhibiting activity.

The features and other details of the invention will now be more particularly described and pointed out in the following examples describing preferred techniques and experimental results. These examples are provided for the purpose of illustrating the invention and should not be construed as limiting.

### EXAMPLES

### Example 1

We performed a multi-centered, open-label, dose-escalation study of a single intravitreous injection of EYE001 in patients with subfoveal CNV secondary to age-related macular degeneration and with a visual acuity worse than 20/200 on the ETDRS chart. The starting dose was 0.25 mg injected once intravitreously. Dosages of 0.5, 1, 2 and 3 mg were also tested. Complete ophthalmic examination with fundus photography and fluorescein angiography was performed. A total of 15 patients were treated.

Selection Criteria.

Patients for the study were selected using the following inclusion and exclusion criteria:

Inclusion Criteria: Patients were required to be >50 years and in generally good health, have a best corrected visual acuity in the study eye worse than 20/200 on the ETDRS chart, and 20/400 or worse for at least the first patient of each cohort (n=3); best corrected visual acuity in the fellow eye equal to or better than 20/64; subfoveal CNV (classic and/or occult CNV) of >3.5 Macular Photocoagulation Study (MPS) disc areas in size; clear ocular media and adequate pupillary dilatation to permit good quality stereoscopic fundus photography; and intraocular pressure of 22 mmHg or less.

Exclusion Criteria: Exclusions included significant media opacities, including cataract, which might interfere with visual acuity, assessment of toxicity, or fundus photography; presence of ocular disease, including glaucoma, diabetic retinopathy, retinal vascular occlusion or other conditions (other than CNV from AMD) which might significantly affect vision; presence of other causes of CNV, including pathologic myopia (spherical equivalent of −8 diopters or more negative), the ocular histoplasmosis syndrome, angioid streaks, choroidal rupture and multifocal choroiditis; patients in whom additional laser treatment for CNV might be indicated or considered; any intraocular surgery within 3 months of study entry; blood occupying >50% of the lesion; previous vitrectomy; previous or concomitant therapy with another investigational agent to treat AMD except multivitamins and trace minerals; any of the following underlying systemic diseases including uncontrolled diabetes mellitus or presence of diabetic retinopathy; cardiac disease including myocardial infarction within 12 months prior to study entry, and/or coronary disease associated with clinical symptoms, and/or indications of ischemia noted on ECG; stroke (within 12 months of study entry); active bleeding disorders; any major surgical procedure within one month of study entry; active peptic ulcer disease with bleeding within 6 months of study entry; and concomitant systemic therapy with corticosteroids (e.g. oral prednisone), or other anti-angiogenic drugs (e.g. thalidomide).

Study Medication.

The drug product was a ready-to-use sterile solution composed of pegaptanib sodium (EYE001 formerly NX1838) dissolved in 10 mM sodium phosphate and 0.9% sodium chloride buffer injection and presented in a sterile and pyrogen free 1 cc glass body syringe barrel, with a coated stopper attached to a plastic plunger, and a rubber end cap on the pre-attached 27 gauge needle. The pegylated aptamer was supplied at active drug concentrations of 1, 2.5, 5, 10, 20 or 30 mg/ml of EYE001 (expressed as oligonucleotide content) in order to provide a 100 μl delivery volume.

Patient Enrollment.

Before recruitment of patients into the study, written Institutional Review Board (IRB) approval of the protocol, informed consent and any additional patient information was obtained.

Results.

A single dose-ranging safety study was performed in 15 patients at doses varying from 0.25 to 3.0 mg/eye without reaching dose-limiting toxicity. Viscosity of the formulation prevented further dose escalation past 3 mg. Patients ranged in age from 64 to 92 years old. Eight males and seven females were entered and all were Caucasian. Eleven of the fifteen patients experienced a total of seventeen mild or moderate, adverse events including six, which were probably or possibly related to administration of EYE001: mild intraocular inflammation, scotoma, visual distortion, hives, eye pain and fatigue. In addition, there was one severe adverse event, which was unrelated to test drug. This was the diagnosis of breast carcinoma in one patient, where the lump had been noted prior to treatment.

At 3 months after injection of EYE001, 12 out of 15 (80%) eyes showed stable or improved vision. Four patients (26.7%) had significantly improved vision at the same time point, which was defined as a 3-line, or greater, increase in vision on the ETDRS chart. Patients with such improved vision at 3 months noted increases of +6, +4 and +3 lines on an ETDRS chart. No unexpected visual safety events were noted. Evaluation of color photographs and fluorescein angiograms revealed no signs of retinal or choroidal toxicity.

Our Phase IA clinical study showed that single intravitreal doses of the anti-VEGF aptamer could be administered safely up to 3 mg/eye. No significant ocular or systemic side effects were noted.

Clinicians agree that a minimum of one-year follow-up is desirable to evaluate any potential treatment for exudative AMD. Nevertheless, 3-month data is available from some prospective studies and is useful to assess both ophthalmic safety and any potential trends of a new therapy.

Historical controls indicate that only 1.4% (pivotal photodynamic trial) (Arch Ophthalmol 1999, 117:1329-1345) and 3.0% (radiation study) (1999, 106;12:2239-2247) of eyes have shown significant visual improvement as defined by a gain of 3 or more lines on an ETDRS chart at 3 months. In addition, the PDT-treated group of the TAP study (Arch Ophthalmol 1999, 117:1329-1345) only noted such improved vision in 2.2% of cases at 3 months. These findings confirm our clinical impression that it is rare to see significant visual improvement at any time frame with any type (classic, occult or mixed) of CNV secondary to AMD.

In our study, at three months after intravitreal administration of the anti-VEGF aptamer, 80% of eyes showed stabilized or improved vision with 26.7% showing an increase in 3 or more lines on the ETDRS chart. These visual improvements are supported by clinical and angiographic findings in some of the aptamer-treated patients. Stabilization of vision has always been the goal of exudative AMD studies, so the significant visual acuity improvement (3 ETDRS lines) seen in 26.7% of patients at 3 months with only one dose was unexpected. Clearly, historical controls are inappropriate for comparison. In addition, the short follow-up period, small sample size, and different CNV type (i.e. percentage of classic, occult, or mixed CNV) precluded any final conclusions or comparisons. However, it appears that the aptamer-treated eyes have certainly shown at least excellent visual safety at 3 months and justify further studies.

Pharmacokinetic Data

Blood was collected over a 28-day period after dosing to obtain serial plasma samples. Pegaptanib concentrations were determined by a dual hybridization assay. EYE001 in human EDTA plasma samples (100 μL) was mixed with two complementary oligonucleotides. One of the oliogonucelotides (capture oligo) was labeled with biotin permitting capture of the complex on a microtiter plate pre-coated with NeutrAvidin. The second was a detection oligo labeled with dioxigenin. The EYE001/oligo mixture was heated to 75° C. and incubated at 37° C. for annealing of the oligos to EYE001. The mixture was then transferred into a Neutr-Avidin coated plate for capture of the complex and detection with an alkaline phosphatase labeled anti-digoxigenin antibody. AttoPhos was used as the substrate for a fluorescent readout. Plasma pegaptanib concentrations were either below or close to the lower limit of quantitation of the assay (7 ng/mL, or 0.007 μg/mL) for doses ≦1.0 mg doses at all time points. Pharmacokinetic parameters values for the 2 and 3 mg/eye dose cohorts are shown in Table 1. In general, plasma concentration-time data were sparse, because pharmacokinetic parameter values were not estimated unless at least 3 measurable plasma concentration-time data points were obtained after administration of a single IVT injection. In addition, pegaptanib Cₘₐₓ and tₘₐₓ were not estimated unless a 24-hour pegaptanib plasma concentration value was available, because there were no data collected between 24 and 168 hours after dosing. Pegaptanib plasma concentrations were measurable within 4 hours after dosing and for up to 29 days after dosing. Maximum pegaptanib concentrations occurred during the first week after pegaptanib administration, probably between 18 hours and 5 days after dosing. Pegaptanib maximum plasma concentration values for the 2.0 and 3.0 mg/eye dose cohorts were between 0.050 and 0.150 μg/mL. The t_{1/2} values, which ranged from 4 to 11 days, should be interpreted with caution, because the data for this study are sparse. The terminal half-life values described here probably represent pegaptanib absorption from the IVT injection site into the systemic circulation rather than drug elimination from the body. Hence, t_{1/2} values should be interpreted with caution.

TABLE 1
Individual Pharmacokinetic Parameters
for the 2 and 3 mg/eye Dose Cohorts

| Subject | | | | | | Terminal |
|---|---|---|---|---|---|---|
| Number | Cₘₐₓ | tₘₐₓ | AUC₍₀₋ₜₗₐₛₜ) | Tₗₐₛₜ | Cₗₐₛₜ | t_{1/2} |
| (mg/eye) | (μg/mL) | (h) | (μg · h/mL) | (days) | (μg/mL) | (days) |
| 0105 (2.0) | 0.098 | 24 | 18 | 29 | 0.007 | 11 |
| 0203 (2.0) | 0.052 | 24 | 9 | 10 | 0.023 | 4 |
| 0204 (2.0) | NC | NC | NC | NC | NC | NC |
| 0205 (3.0) | 0.148 | 24 | 36 | 28 | 0.010 | 7 |
| 0304 (3.0) | 0.070 | 168 | 28 | 28 | 0.017 | 10 |
| 0305 (3.0) | NC | NC | 17 | 28 | 0.050 | NC |

NC = not calculated because plasma concentration data do not decline in a log-linear fashion after the maximum concentration is reached or the data are too sparse. Cₘₐₓ or tₘₐₓ is not reported if subject did not contribute a 24-hour sample.

Levels of human anti-pegaptanib sodium IgG were measured using a sandwich enzyme immunoassay prior to and at Days 7, 14, and 28 following administration of the pegylated aptamer. The immunoassay uses a surrogate rabbit anti-EYE001 to estimate human anti-EYE001 in human serum samples. Two different biotinylated detect antibodies are used, goat rabbit IgG and donkey anti-human IgG. EYE001 was coated on a polystyrene 96 well microplate, which was blocked to minimize nonspecific binding. Reference calibrator (human IgG), normal and positive controls (rabbit anti-EYE001) and human serum samples were added to the plate. Biotinylated goat anti-rabbit IgG and donkey anti-human IgG and Strtavidin conjugated to HRP were serially added to specific wells on the plate. TMB-HRP was added to the substrate, which produces a color that is directly proportional to the relative concentration of anti-EYE001 in the samples. No antibodies were detected at any dose.

In summary, pre-clinical and early clinical results with single intravitreal injections of the anti-VEGF aptamer are very encouraging. The safety of single-dose intravitreal injections of dosages up to 3 mg/eye has been established.

### Example 2

We conducted a multi-center, open-label, repeat dose Phase IB study of 3 mg/eye of pegaptanib sodium (EYE001, an anti-VEGF aptamer) in patients with subfoveal CNV secondary to AMD with a visual acuity worse than 20/100 in the study eye and better or equal to 20/400 in the fellow eye. If 3 or more patients experienced Dose-Limiting Toxicity (DLT's), the dose was reduced to 2 mg and then 1 mg, if necessary. The intended number of patients to be treated was 20; 10 patients with the anti-VEGF aptamer alone and 10 patients with both anti-VEGF therapy and PDT. Eleven sites in the U.S. were selected for the studies.

Selection Criteria.

Patients for the study were selected using the following inclusion and exclusion criteria:

Inclusion Criteria: The ophthalmic criteria included best corrected visual acuity in the study eye worse than 20/100 on the ETDRS chart, best corrected visual acuity in the fellow eye equal to or better than 20/400, subfoveal choroidal neovascularization with active CNV (either classic and/or occult) of less than 12 total disc areas in size secondary to age related macular degeneration, clear ocular media and adequate pupillary dilatation to permit good quality stereoscopic findus photography, and intraocular pressure of 21 mmHg or less. General criteria included patients of either sex, aged ≧50 years; performance Status ≦2 according to the Eastern Cooperative Oncology Group (ECOG)/World Health Organization (WHO) scale, normal electrocardiogram (ECG) or clinically non-significant changes; women must be using an effective contraceptive, be post-menopausal for at least 12 months prior to study entry, or surgically sterile; if not, a serum pregnancy test must be performed within 48 hours prior to treatment and the result made available prior to treatment initiation, an effective form of contraceptive should be implemented for at least 28 days following the last dose of EYE001; adequate hematological function: hemoglobin ≧10 g/dl; platelet count ≧150×10⁹/l; WBC ≧4×10⁹/l; PTT within normal range of institution; adequate renal function: serum creatinine and BUN within 2× the upper limit of normal (ULN) institution; adequate liver function: serum bilirubin ≦1.5 mg/dl; SGOT/ALT, SGPT/AST, and alkaline phosphatase within 2× ULN of institution; written informed consent; and ability to return for all study visits.

Exclusion Criteria: Patients were not eligible for the study if any of the following criteria were present in the study eye or systemically: patients scheduled to receive, or have received any prior Photodynamic Therapy with Visudyne; significant media opacities, including cataract, which might interfere with visual acuity, assessment of toxicity or fundus photography; presence of other causes of choroidal neovascularization, including pathologic myopia (spherical equivalent of −8 diopters or more negative), the ocular histoplasmosis syndrome, angioid streaks, choroidal rupture and multifocal choroiditis; patients in whom additional laser treatment for choroidal neovascularization might be indicated or considered; any intraocular surgery within 3 months of study entry; previous vitrectomy; previous or concomitant therapy with another investigational agent to treat AMD except multivitamins and trace minerals; previous radiation to the fellow eye with photons or protons; known allergies to the fluorescein dye used in angiography or to the components of EYE001 formulation; any of the following underlying systemic diseases including: uncontrolled diabetes mellitus or presence of diabetic retinopathy, cardiac disease: myocardial infarction within 12 months prior to study entry, and/or coronary disease associated with clinical symptoms, and/or indications of ischemia noted on ECG, impaired renal or hepatic function, stroke (within 12 months of study entry), active infection, active bleeding disorders, any major surgical procedure within one month of study entry, active peptic ulcer disease with bleeding within 6 months of study entry; concomitant systemic therapy with corticosteroids (e.g. oral prednisone), or other anti-angiogenic drugs (e.g. thalidomide); previous radiation to the head and neck; any treatment with an investigational agent in the past 60 days for any condition; any diagnosis of cancer in the past 5 years, with the exception of basal or squamous cell carcinoma.

Study Medication.

Drug Supply

EYE001 was used as the anti-VEGF therapy in this study. EYE001 drug substance is a pegylated anti-VEGF aptamer. It was formulated in phosphate buffered saline at pH 5-7. Sodium hydroxide or hydrochloric acid may be added for pH adjustment.

EYE001 was formulated at three different concentrations: 3 mg/100 ul, 2 mg/100 ul and 1 mg/100 ul packaged in a sterile 1 ml, USP Type I graduated glass syringe fitted with a sterile 27-gauge needle. The drug product was preservative-free and intended for single use by intravitreous injection only. The product was not used if cloudy or particles were present.

The active ingredient was EYE001 Drug Substance, (Pegylated) anti-VEGF aptamer, and 30 mg/ml, 20 mg/ml and 10 mg/ml concentrations. The excipients were Sodium Chloride, USP; Sodium Phosphate Monobasic, Monohydrate, USP; Sodium Phosphate Dibasic, Heptahydrate, USP; Sodium Hydroxide, USP; Hydrochloric acid, USP; and Water for injection, USP.

Dose and Administration

Preparation. The drug product was a ready-to-use sterile solution provided in a single-use glass syringe. The syringe was removed from refrigerated storage at least 30 minutes (but not longer than 4 hours) prior to use to allow the solution to reach room temperature. Administration of the syringe contents involved attaching the threaded plastic plunger rod to the rubber stopper inside the barrel of the syringe. The rubber end cap was then removed to allow administration of the product.

Treatment Regimen and Duration. EYE001 was administered as a 100 μl intravitreal injections on three occasions at 28 day intervals. Patients were enrolled to receive 3 mg/injection. If 3 or more patients experienced Dose-Limiting Toxicity (DLT's), the dose was reduced to 2 mg and further to 1 mg, if necessary, each in an additional 10 patients.

PDT Administration.

PDT was given with EYE001 only in cases with predominantly classic CNV. The standard requirements and procedures for PDT administration were used as described in Arch Ophthalmol 1999, 117:1329-1345. PDT was required to be given 5-10 days prior to administration of the anti-VEGF aptamer.

Patient Enrollment.

Before recruitment of patients into the study, written Institutional Review Board (IRB) approval of the protocol, and informed consent form were obtained. Case report form screening pages were completed by study site personnel. Patients who meet the eligibility criteria and have provided written informed consent were enrolled in the study.

Follow-up Schedule.

Patients were clinically evaluated by the ophthalmologist several days after injection and again one-month later just prior to the next injection. ETDRS visual acuities, kodachrome photography and fluorescein angiography were performed monthly for the first 4 months.

Endpoints.

The safety parameters given under the DLT section above were the primary endpoint of the studies. In addition, the percentage of patients with stabilized (0 line change or better) or improved vision at 3 months, the percentage of patients with a 3-line or greater improvement at 3 months, and the need for PDT re-treatment at 3 month as determined by the investigator were other endpoints studied.

### Results

TABLE 2
Visual data of patients with subfoveal CNV
treated with anti-VEGF aptamer alone.

| | | | | | | ± No of |
|---|---|---|---|---|---|---|
| | | | | | | Lines |
| Patient | Base- | | | | | At |
| # | line | Day 8 | Day 29 | Day 57 | Day 85 | Day 85 |
| 03-001 | 20/50 | 20/40 | 20/40 | 20/32 | 20/32 | +2 |
| 04-001 | 20/125 | 20/64 | 20/80 | 20/80 | 20/80 | +2 |
| 06-001 | 20/160 | 20/125 | 20/100 | 20/125 | OUT | +1 |
| 07-001 | 20/100 | 20/100 | 20/64 | 20/80 | 20/80 | +1 |
| 07-002 | 20/320 | 20/80 | 20/64 | 20/64 | 20/50 | +8 |
| 08-001 | 20/125 | 20/125 | 20/100 | 20/100 | 20/160 | −1 |
| 09-001 | 20/500 | 20/200 | 20/400 | 20/320 | OUT | +2 |
| | | | | (Day | | |
| | | | | 36) | | |
| 10-001 | 20/500 | 20/640 | 20/500 | 20/400 | 20/500 | 0 |
| 10-002 | 20/200 | 20/125 | 20/160 | 20/160 | 20/160 | +1 |
| 10-003 | 20/400 | 20/160 | 20/160 | 20/160 | 20/126 | +5 |

### Change in Vision at 3 Months

| | Stabilized or | ≧3 Line |
|---|---|---|
| | Improved | Improvement |
| EYE001 Treated - | 87.5% | 25.0% |
| (N = 8) which | | |
| represents all eyes | | |
| that completed | | |
| the protocol. | | |

Eleven patients were treated with both the anti-VEGF aptamer and PDT. In this group of patients (N=10) who completed the 3-month treatment regimen, 90% had stabilized or improved vision and 60% showed a 3-line improvement of vision on the ETDRS chart at 3 months (Table 3). These 3-line improvements included gains of +3, +5, +4, +4, +6, and +3 ETDRS lines of vision.

TABLE 3
Visual data of patients with subfoveal CNV treated
with anti-VEGF aptamer combined with PDT.

| | | | | | | | ± No of Lines |
|---|---|---|---|---|---|---|---|
| Patient | | | | | | Repeat | At latest time- |
| # | Baseline | Day 8 | Day 29 | Day 57 | Day 85 | PDT | point |
| 06-011 | 20/400 | 20/320 | 20/100 | 20/640 | 20/200 | NO | +3 |
| 06-012 | 20/250 | 20/160 | 20/125 | 20/125 | 20/80 | NO | +5 |
| 08-011 | 20/40 | 20/32 | 20/20 | 20/20 | 20/26 | YES | +2 |
| 10-011 | 20/160 | 20/160 | 20/160 | 20/160 | OUT | NO | 0 |
| 05-011 | 20/100 | 20/64 | 20/64 | 20/64 | 20/40 | NO | +4 |
| 12-011 | 20/160 | 20/100 | 20/250 | 20/200 | 20/200 | NO | −1 |
| 06-013 | 20/800 | 20/640 | 20/800 | 20/800 | 20/320 | YES | +4 |
| 02-011 | 20/500 | 20/200 | 20/160 | 20/80 | 20/126 | YES | +6 |
| 06-014 | 20/100 | 20/80 | 20/80 | 20/80 | 20/100 | NO | 0 |
| 06-015 | 20/125 | 20/40 | 20/64 | 20/50 | 20/80 | NO | +2 |
| 02-012 | 20/500 | 20/500 | 20/125 | 20/320 | 20/250 | YES | +3 |

### Change in Vision at 3 Months

| | Stabilized or | ≧3 Line |
|---|---|---|
| | Improved | Improvement |
| EYE001 Treated - | 90% | 60% |
| (N = 10) which | | |
| represents all eyes | | |
| that completed the | | |
| protocol. | | |

Of the remaining patients who did not show a 3-line gain, only one showed a loss of vision at 3 months and this patient lost only one line of vision at this time point. No patient in this group lost more than one line of vision at 3 months.

Repeat PDT treatment at 3 months (whose need was solely determined by the investigator) was performed in 4 of 10 eyes (40%) that participated for the complete duration of the study.

Pharmacokinetic Data

Without PDT:

Subjects received 3 consecutive unilateral, IVT injections of 3 mg pegaptanib sodium/eye at 28-day intervals. Serial blood samples were taken for the determination of pegaptanib plasma concentrations after the 3 IVT injections. Pegaptanib plasma concentrations were measured by a validated dual-hybridization assay with a lower limit of quantitation of <8 ng/mL (0.008 μg/mL) as described above

Pegaptanib pharmacokinetic parameter values as a function of dosing interval are shown in Table 4. Pegaptanib plasma concentrations were measurable within 2 to 6 hours after IVT administration of 3 mg of pegaptanib sodium per study eye. Maximum pegaptanib plasma concentrations occurred during the first week after administration and then declined over the next 4 weeks to concentrations that approached the lower limit of quantitation of the assay. IVT injections of 3 mg pegaptanib sodium every 28 days resulted in no apparent accumulation of pegaptanib in plasma. The mean t_{1/2} of pegaptanib in plasma ranges from 7 to 12 days. The t_{1/2} values described here probably represent pegaptanib absorption from the IVT injection site into the systemic circulation rather than drug elimination from the body.

Multiple dose treatment with 3 mg of pegaptanib sodium, given IVT every 28 days for a total of 3 doses did no induce anti-pegaptanib IgG-mediated antibody production using the immunoassay described above.

TABLE 4
Mean ± SD (Range) Pegaptanib Plasma Pharmacokinetic Parameters in Subjects Receiving 3 mg/eye Every 28 Days for 3 Months

| | | | | | | | | Terminal |
|---|---|---|---|---|---|---|---|---|
| | tₘₐₓ | Cₘₐₓ | Tₗₐₛₜ | Cₗₐₛₜ | AUC₍₀₋ₜₗₐₛₜ₎ | AUC_{(0-∞)} | AUCτ | t_{1/2} |
| | (h) | (μg/mL) | (h) | (μg/mL) | (μg · h/mL) | (μg · h/mL) | (μg · h/mL) | (days) |
| First Dose | 39 ± 45 | 0.083 ± 0.032 | 440 ± 81 | 0.012 ± 0.003 | 16 ± 5 | 20 ± 6 | NC | 7 ± 3 |
| (N = 10) | (23-168) | (0.033-0.124) | (335-529) | (0.009-0.021) | (10-25) | (12-29) | | (3-13) |
| Second Dose^{a} | 38 ± 54 | 0.070 ± 0.027 | 176 ± 12^{a} | 0.036 ± 0.009^{a} | 9 ± 3^{a} | NC | NC | NC |
| (N = 10) | (4-189) | (0.032-0.109) | (165-192) | (0.019-0.051) | (4-15) | | | |
| Third Dose | 38 ± 48 | 0.087 ± 0.052 | 490 ± 318 | 0.021 ± 0.015 | 17 ± 10 | NC | 24 ± 7 | 12 ± 4 |
| (N = 9)^{b} | (22-166) | (0.039-0.200) | (166-1005) | (0.008-0.046) | (5-34) | | (17-34) | (8-17) |

^{a}Samples were taken only up to 1 week after the second injection and then just prior to the third injection; PK parameters Tₗₐₛₜ, Cₗₐₛₜ, and AUCₗₐₛₜ are not estimated reliably.
^{b}One subject did not receive all 3 injections because he withdrew consent before the last injection.

With PDT:

Serial blood samples were taken after the first, second, and third IVT injections of 3 mg per study eye of pegaptanib sodium for the determination of pegaptanib plasma concentrations. Pegaptanib plasma concentrations were measured by a validated dual hybridization assay described above. Pegaptanib plasma pharmacokinetic parameters were calculated by noncompartmental methods.

Pegaptanib plasma concentrations were measurable within 2 to 6 hours in most subjects receiving an IVT injection of 3 mg pegaptanib sodium (Table 5). Pegaptanib plasma concentrations are maximum during the first week after the injection and then decline over 4 weeks to values that approach the lower limit of sensitivity of the assay (<8 ng/mL). IVT injections of 3 mg pegaptanib sodium every 28 days result in no apparent accumulation of pegaptanib in plasma. The mean terminal half-life of pegaptanib in plasma ranges from 8 to 10 days. The terminal half-life values described here represent pegaptanib absorption from the IVT injection site into the systemic circulation rather than drug elimination from the body.

TABLE 5
Mean ± SD (Range) Pegaptanib Plasma Pharmacokinetics

| Pharmacokinetic | First Dose | | Second Dose^{a} | | Third Dose^{b} | |
|---|---|---|---|---|---|---|
| Parameter | (N = 11) | | (N = 11) | | (N = 10) | |
| Tₘₐₓ (h) | 24 ± 2 | (22-29) | 39 ± 51 | (4-163) | 21 ± 7 | (4-26) |
| Cₘₐₓ (μg/mL) | 0.068 ± 0.026 | (0.031-0.119) | 0.067 ± 0.035 | (0.030-0.133) | 0.074 ± 0.040 | (0.032-0.152) |
| Tₗₐₛₜ (h) | 482 ± 65 | (343-529) | 189 ± 52^{a} | (168-314) | 395 ± 267 | (168-695) |
| Cₗₐₛₜ (μg/mL) | 0.013 ± 0.004 | (0.008-0.021) | 0.033 ± 0.011^{a} | (0.020-0.054) | 0.022 ± 0.015 | (0.008-0.051) |
| AUC₍₀₋ₜₗₐₛₜ₎ | 16 ± 4 | (10-22) | 9 ± 4^{a} | (4-14) | 17 ± 12 | (5-41) |
| (μg · h/mL) | | | | | | |

| AUC_{(0-∞)} | 21 ± 5 | (12-27) | NC | NC | |
|---|---|---|---|---|---|
| (μg · h/mL) | | | | | |
| AUCₜₐᵤ(μg · h/mL) | NC | | NC | 25 ± 12 | (13-41) |
| Terminal t_{1/2} | 10 ± 3 | (6-14) | NC | 8 ± 4 | (2-12) |
| (days) | | | | | |
| CL/F (mL/h) | 152 ± 41 | (112-249) | NC | 143 ± 67 | (74-234) |

NC = not calculated
^{a}Samples were taken only up to 1 week after the second injection and then just prior to the third injection; PK parameters of Cₗₐₛₜ, Tₗₐₛₜ, AUC are therefore not estimated reliably.
^{b}One subject did not receive all 3 injections because he withdrew consent before the last injection. For the first dose, CL/F = Dose/AUC_{(0-∞)}, and for the third dose CL/F = Dose/AUCtau.

Mean AUC₍₀₋ₜₗₐₛₜ₎ values for the first and third injection were similar, as were AUC_{(0-∞)} values for the first injection versus AUC_{(0-τ,ss)} for the third injection, suggesting no apparent accumulation of pegaptanib in plasma upon multiple dosing.

The terminal half-life values ranged from 2 to 14 days with mean values ranging from 8 to 10 days. It is not possible to compare reliably the terminal half-lives from the first and last injection due to the limited data.

The apparent clearance (Cl/F) values of pegaptanib after IVT administration ranged from 74 to 249 mL/h and are low relative to renal and hepatic plasma flow rates.

### Example 3

Pegaptanib sodium drug substance, a pegylated (40 kD) anti-VEGF aptamer (the anti-VEGF pegylated aptamer EYE001) was used. As discussed above, this aptamer is a polyethylene glycol (PEG)-conjugated oligonucleotide that binds to the major soluble human VEGF isoform with high specificity and affinity. The aptamer binds and inactivates VEGF in a manner similar to that of a high-affinity antibody directed towards VEGF.

The pegylated pegaptanib sodium drug substance was formulated in phosphate buffered saline at pH 5-7. Sodium hydroxide or hydrocholoric acid was added for pH adjustment.

The aptamer was formulated as 0.3, 1.0 and 3 mg/100 μl and packaged in a sterile 1 ml USP Type I graduated glass syringe fitted with a sterile 27 gauge needle. The syringe contents were allowed to reach room temperature before use and subsequently administered as a 100 μl intravitreal injection every 6 weeks for 54 weeks.

Patients were randomized to one of 4 treatment groups (0.3 mg pegaptanib sodium/eye, 1 mg pegaptanib sodium/eye, 3 mg pegaptanib sodium/eye or sham injections) and received a total of 9 intravitreous or sham injections once every 6 weeks for 48 weeks with a follow up period to 54 weeks. Patients were stratified by center, by percentage of classic CNV (visible classic CNV portion divided by total lesion area) [predominantly classic (≧50% classic CNV), minimally classic (1-49% classic CNV), occult with no classic (0% classic CNV)], and according to whether or not they had received prior photodynamic therapy (PDT) (no more than once). Patients in the active therapy arms were re-randomized at week 54 (1:1) to either discontinue or continue the study for a further 48 weeks (8 injections). Those patients receiving sham injections were re-randomized at week 54 on a 1:1:1:1:1 basis to discontinue the study, to continue on study receiving one of the 3 active treatments, or to continue on sham therapy. Primary evaluations in the first study year occurred at each treatment visit, with assessment of change in visual acuity (VA) at weeks 6, 12 and 54, change in CNV status and quality-of-life scores at weeks 30 and 54, and pharmacokinetic assessments at weeks 12, 30, 42 and 54.

1% Mydriacyl and 2.5% Phenylephrine were applied topically to the study eye to achieve adequate pupillary dilation. Two to three drops of 50% saline diluted 10% povidone-iodine (betadine) solution were instilled into the eye. In the event of allergy to iodine, a drop of topical antibiotic was placed on the conjunctiva in place of iodine. A subconjunctival injection of 0.5 ml 2% xylocaine without epinephrine was administered in the inferotemporal quadrant in all patients—3.0 to 3.5 mm from the limbus in aphakic/pseudophakic patients, and 3.5 to 4.0 mm in phakic patients. Investigators were instructed to select one of two pre-injection procedures (Options A and B, below). For patients with iodine allergy, investigators were required follow Option A, instilling one additional drop of antibiotic instead of povidone-iodine.
A. Administer topical ofloxacin, levofloxacin, or an antibiotic drop with comparable antimicrobial coverage for three days prior to the treatment followed by three consecutive drops of antibiotic and several drops of 5% povidone-iodine immediately before the treatment
B. Administer three consecutive drops of antibiotic and a 5% povidone-iodine flush of the fornices and caruncle with at least 10 cc of solution just prior to treatment.

Prior to treatment, topical antibiotic drops were administered 3 times separated by at least 5 minutes within one hour prior to treatment.

For patients who were prepared under Option A, following the last dose of antibiotic, the investigator instilled two or three drops of 5% povidone-iodine into the eye. Using sterile gloves and cotton-tip applicators soaked in 5% povidone iodine, the investigator scrubbed the eyelids, the upper and lower eyelid margins, and the caruncle 3 times. In the event of allergy to iodine, one additional drop of antibiotic was instilled instead of povidone-iodine.

For patients who were prepared under Option B, the investigator waited at least 5 minutes after the last dose of antibiotic to perform a 5% povidone-iodine flush, irrigating the fornices and the caruncle with at least 10 cc of 5% povidone-iodine using a forced stream from a syringe connected to an angio-catheter to effect mechanical debridement.

After changing gloves, the investigator isolated the ocular field with a drape, pinning the eyelashes to the eyelids, and placed one or two drops of 5% povidone-iodine on the ocular surface at the intended treatment site. An eyelid speculum had to be used for all injections.

Treatment Administration—Pegaptanib Sodium or Sham Injection:

Active Drug: For patients randomized to receive injections of study drug, following the administration of subconjunctival xylocaine, the rubber stopper covering the needle was removed and the entire volume of the drug was injected. The needle of the pegaptanib sodium syringe was inserted until the tip was just visualized through the dilated pupil.

Sham: For patients randomized to receive the control injection, following the administration of subconjunctival xylocaine, the investigator used the sterile empty syringe that was provided with a cover but without an attached needle. The blunt opening of the syringe barrel was used to indent the conjunctiva in the inferotemporal quadrant to simulate the pressure on the eyeball of an injection. The patients were instructed to look away prior to the injection and the procedure was performed in a manner so that the patient was not aware of the use of a needleless syringe and the lack of penetration of the globe.

Pharmacokinetic Analyses

In the Examples, the pharmacokinetic parameter, Cₘᵢₙ, for each patient was reported as the observed pegaptanib plasma concentration in samples taken before the injection of study drug at weeks 12, 30, 42 and 54. As it was anticipated that steady-state would be reached by week 12. An average of these trough or minimum concentrations were reported as the C_{ave,min} value for each patient.

Pharmacokinetic parameters were estimated using individual profiles of pegaptanib plasma concentrations in patients participating in the nested pharmacokinetic study and included the following: the observed maximum pegaptanib plasma concentration (Cₘₐₓ) and the time of this maximum concentration (Tₘₐₓ) were determined. The slope of the terminal log-linear portion of each profile was determined by least squares linear regression, and the terminal phase half-life (t_{1/2}) was calculated by dividing 0.693 by this slope. The linear trapezoidal method was used to determine the area under the plasma concentration-time curve from time zero to infinite time after the first dose (AUC_{inf}) or from time zero to 6 weeks (AUCₜₐᵤ, where tau is the dosing interval of 6 weeks). The apparent total body clearance (Cl/F) was estimated as Dose/AUC_{inf} for the first dose and as CL/F=Dose/AUCₜₐᵤ for subsequent doses. If patients did not donate a blood sample at 24 hours, individual Cmax, Tmax and AUC values are reported but are not included in the descriptive statistics for that dose because estimates of these pharmacokinetic parameters in the absence of the 24 hour plasma concentration data are unreliable as samples were not collected between 24 hours and the 1 week (168 hours) sample.

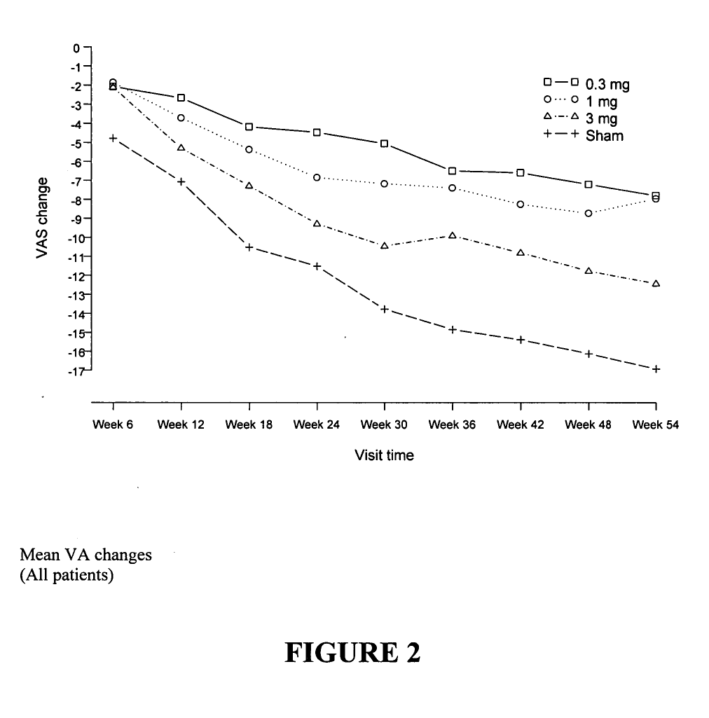

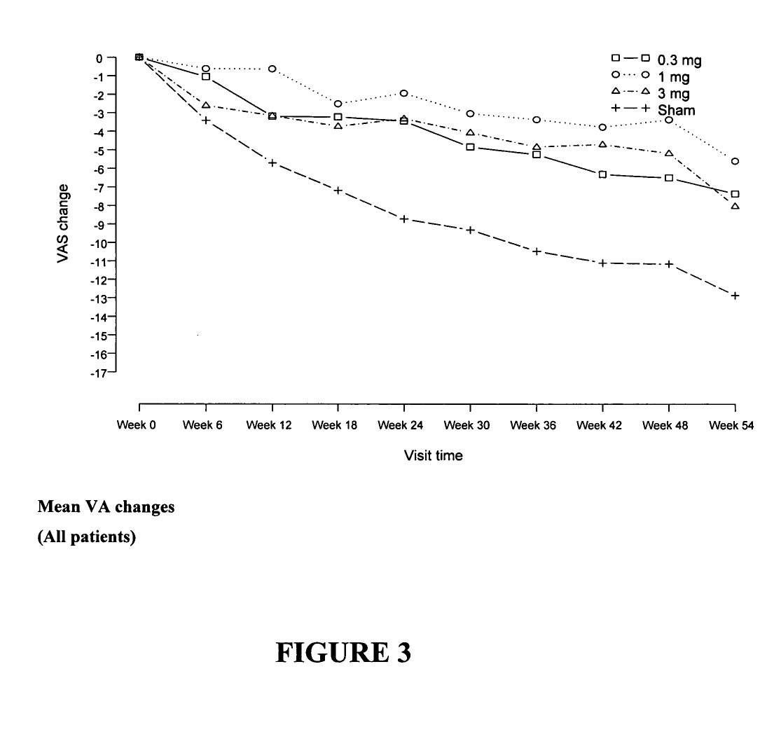

The results are shown in Tables 6-27 and FIGS. 2-3 below. Efficacy was demonstrated for all three doses with no statistical evidence of differential response by dose. The onset of efficacy was evident as early as six weeks post treatment and increased over time up to week 54, as measured by mean visual acuity loss from baseline compared to sham. For 0.3 mg pegaptanib, 70% of patients lost <15 letters of visual acuity (VA) versus 55% for controls. The risk of severe VA loss (≧30 letters; approximately 6 lines) was reduced from 22% to 10% (P<0.0001; 0.3 mg vs. sham). Compared with sham, more patients receiving 0.3 mg pegaptanib maintained or gained VA (33% vs. 23%; P=0.003). As early as 6 weeks, and at all subsequent time points, mean VA was better in the pegaptanib-treated patients (P<0.002, 0.3 mg vs. sham at each time point). There was no evidence that baseline angiographic subtype, lesion size or initial level of VA precluded a treatment benefit. These visual results were further confirmed using a masked angiographic measurements, which revealed a reduction in total lesion size, choroidal neovascularization size, and area of leakage. Pegaptanib was generally well tolerated at all doses. Serious adverse events such an endophthalmitis (infection), traumatic lens injury and retinal detachment were infrequent (1.3%, 0.6% or patients, respectively), were attributed by investigators to the injection procedure rather than to the study drug, and rarely were associated with severe VA loss (≦0.1% of patients).

TABLE 6
Loss of ≧ 30 letters
(Predominantly classic)

| Arm | % losing ≧ 15 letters |
|---|---|
| 3 MG | 103/296 (35%) |
| 1 MG | 87/300 (29%) |
| 0.3 MG | 88/294 (30%) |
| Controls | 132/296 (45%) |

TABLE 7
Loss of ≧ 15 letters
(All patients)

| Arm | % losing ≧ 15 letters |
|---|---|
| 3 MG | 47/153 (31%) |
| 1 MG | 38/154 (25%) |
| 0.3 MG | 41/150 (27%) |
| Controls | 63/152 (41%) |

TABLE 8
Loss of ≧ 15 letters
(All patients)

| Arm | % losing ≧ 15 letters |
|---|---|
| 3 MG | 56/143 (39%) |
| 1 MG | 49/146 (34%) |
| 0.3 MG | 47/144 (33%) |
| Controls | 69/144 (48%) |

TABLE 9
Stable or gain > 0 letters
(All patients)

| Arm | % gaining ≧ 0 letters |
|---|---|
| 3 MG | 93/296 (31%) |
| 1 MG | 110/300 (37%) |
| 0.3 MG | 98/294 (33%) |
| Controls | 68/296 (23%) |

TABLE 10
Stable or gain > 0 letters
(All patients)

| Arm | % gaining ≧ 0 letters |
|---|---|
| 3 MG | 33/143 (23%) |
| 1 MG | 51/146 (35%) |
| 0.3 MG | 49/144 (34%) |
| Controls | 25/144 (17%) |

TABLE 11
Stable or gain > 0 letters
(All patients)

| Arm | % gaining ≧ 0 letters |
|---|---|
| 3 MG | 60/153 (39%) |
| 1 MG | 59/154 (38%) |
| 0.3 MG | 49/150 (33%) |
| Controls | 43/152 (28%) |

TABLE 12
Gain of ≧ 15 letters
(All patients)

| Arm | % gaining ≧ 15 letters |
|---|---|
| 3 MG | 13/296 (4%) |
| 1 MG | 20/300 (7%) |
| 0.3 MG | 18/294 (6%) |
| Controls | 6/296 (2%) |

TABLE 13
Gain of ≧ 15 letters
(All patients)

| Arm | % gaining ≧ 15 letters |
|---|---|
| 3 MG | 6/143 (4%) |
| 1 MG | 10/146 (7%) |
| 0.3 MG | 12/144 (8%) |
| Controls | 1/144 (1%) |

TABLE 14
Gain of ≧ 15 letters
(All patients)

| Arm | % gaining ≧ 15 letters |
|---|---|
| 3 MG | 7/153 (5%) |
| 1 MG | 10/154 (6%) |
| 0.3 MG | 6/150 (4%) |
| Controls | 5/152 (3%) |

TABLE 15
Combined EOP1003 & 1004
Other endpoint - Loss of ≧ 30 letters
(All patients)

| Arm | % losing ≧ 30 letters |
|---|---|
| 3 MG | 40/296 (14%) |
| 1 MG | 24/300 (8%) |
| 0.3 MG | 28/294 (10%) |
| Controls | 65/296 (22%) |

TABLE 16
Combined EOP1003 & 1004
Secondary endpoint - Mean 54 wk VA change
(All patients)

| | Mean VA change |
|---|---|
| Arm | (Letters Lost) |
| 3 MG | −9.67 |
| 1 MG | −7.21 |
| 0.3 MG | −7.82 |
| Controls | −15.20 |

TABLE 17
EOP1004 (North America Trial)
Secondary endpoint - Mean 54 wk VA change
(All patients)

| Arm | Mean VA change |
|---|---|
| 3 MG | −12.55 |
| 1 MG | −8.50 |
| 0.3 MG | −7.56 |
| Controls | −17.55 |

TABLE 18
EOP1003 (Ex-North America Trial)
Secondary endpoint - Mean 54 wk VA change
(All patients)

| Arm | Mean VA change |
|---|---|
| 3 MG | −6.96 |
| 1 MG | −5.90 |
| 0.3 MG | −7.53 |
| Controls | −13.02 |

TABLE 19
Combined EOP1003 & 1004
Secondary endpoint - Mean 54 wk VA change
(Occult)

| Arm | Mean VA change |
|---|---|
| 3 MG | −9.59 |
| 1 MG | −6.08 |
| 0.3 MG | −9.02 |
| Controls | −16.48 |

TABLE 20
Combined EOP1003 & 1004
Primary endpoint - Responders
(Loss of < 15 letters)
(Occult)

| Arm | % losing < 15 letters |
|---|---|
| 3 MG | 75/111 (68%) |
| 1 MG | 84/116 (72%) |
| 0.3 MG | 74/112 (66%) |
| Controls | 68/120 (57%) |

TABLE 21
Combined EOP1003 & 1004
Secondary endpoint - Stable or gain > 0 letters
(Occult)

| Arm | % gaining ≧ 0 letters |
|---|---|
| 3 MG | 36/111 (32%) |
| 1 MG | 52/116 (45%) |
| 0.3 MG | 39/112 (35%) |
| Controls | 22/120 (18%) |

TABLE 22
Combined EOP1003 & 1004
Secondary endpoint - Gain of ≧ 15 letters
(Occult)

| Arm | % gaining ≧ 15 letters |
|---|---|
| 3 MG | 6/111 (5%) |
| 1 MG | 10/116 (9%) |
| 0.3 MG | 10/112 (9%) |
| Controls | 0/120 (0%) |

TABLE 23
Combined EOP1003 & 1004
Secondary endpoint - Mean 54 wk VA change
(Minimally Classic)

| Arm | Mean VA change |
|---|---|
| 3 MG | −9.52 |
| 1 MG | −6.52 |
| 0.3 MG | −7.39 |
| Controls | −14.33 |

TABLE 24
Combined EOP1003 & 1004
Primary endpoint - Responders
Loss of < 15 letters
(Minimally classic)

| Arm | % losing < 15 letters | |
|---|---|---|
| 3 MG | 73/105 | (70%) |
| 1 MG | 79/106 | (75%) |
| 0.3 MG | 82/109 | (75%) |
| Controls | 52/99 | (53%) |

TABLE 25
Combined EOP1003 & 1004
Secondary endpoint - Mean 54 wk VA change
(Predominantly Classic)

| Arm | Mean VA change |
|---|---|
| 3 MG | −10.41 |
| 1 MG | −10.06 |
| 0.3 MG | −7.07 |
| Controls | −13.88 |

TABLE 26
Combined EOP1003 & 1004
Primary endpoint - Responders
Loss of < 15 letters
(Predominantly classic)

| Arm | % losing < 15 letters |
|---|---|
| 3 MG | 45/80 (56%) |
| 1 MG | 50/78 (64%) |
| 0.3 MG | 50/73 (69%) |
| Controls | 43/76 (57%) |

TABLE 27
Combined EOP1003 & 1004
Other endpoint - Loss of ≧ 30 letters
(Predominantly classic)

| Arm | % losing ≧ 30 letters |
|---|---|
| 3 MG | 11/80 (14%) |
| 1 MG | 6/78 (8%) |
| 0.3 MG | 4/73 (5%) |
| Controls | 18/76 (24%) |

1. A method for treating macular degeneration in a patient comprising:
(a) administering a therapeutically effective amount of the anti-VEGF aptamer identified by the following structure 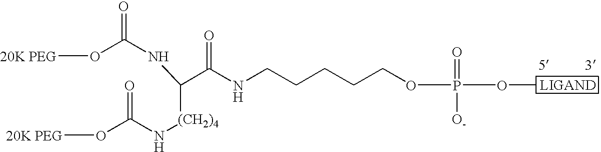
Ligand Component=fCmGmGrArAfUfCmAmGfUmGmAmAfUmGfCfUfUmAfUmAfCmAfUfCfCmG-3′3′−(VEGF ligand)
locally into the eye; and
(b) providing said patient with phototherapy,
wherein the treatment is effective to treat occult, minimally classic, and predominantly classic forms of wet macular degeneration.
